(19) United States
(12) Patent Application Publication
(10) Pub. No.: US 2020/0397710 A1
(43) Pub. Date: Dec. 24, 2020

(54) MAGNETOELECTRIC NANOPARTICLES FOR ACUPUNCTURE TREATMENT OF DISEASES

(71) Applicants: Sakhrat Khizroev, (US); The Trustees of Indiana University, Indianapolis, IN (US)

(72) Inventors: Xiaoming Jin, Carmel, IN (US); Tyler Nguyen, Fishers, IN (US); Xiaomei Shao, Indianapolis, IN (US); Sakhrat Khizroev, Miami, FL (US)

(73) Assignee: The Trustees of Indiana University, Indianapolis, IN (US)

(21) Appl. No.: 16/747,894

(22) Filed: Jan. 21, 2020

Related U.S. Application Data
(60) Provisional application No. 62/793,929, filed on Jan. 18, 2019.

(51) Int. Cl. A61K 9/51; A61K 33/26; A61N 2/02

(52) U.S. Cl. CPC A61K 9/51; B82Y 5/00; A61N 2/02; A61K 33/26

(57) ABSTRACT

The present invention introduces magnetoelectric nanoparticles into acupuncture points that interact with cells, receptors, or molecules in the presence of an applied magnetic field to treat various disorders.

This invention relates to magnetoelectric nanoparticles (MENs) that interact with cells and receptors in the presence of an applied magnetic field to treating various disorders.

Conventional acupuncture is a technique in which practitioners stimulate specific points on a subject's body by inserting a plurality of metallic needles at selected points to treat various disorders. Conventional acupuncture is used to treat myriad disorders including neurological disorders such as pain, epilepsy, and stroke. Problems associated with conventional acupuncture include pain from needle insertion, temporary post-treatment soreness, potential infection, blood vessel injury, internal organ injury and need for repeated visits to an acupuncture practitioner.

A need remains for improved treatment methods that avoid the drawbacks of conventional acupuncture. Also, a need remains to provide non-invasive methods to treat neurological disorders such as neuropathic pain, stroke, and acquired epilepsy. Accordingly, this invention uses certain magnetoelectric nanoparticles that interact with cells and tissues in the presence of an applied magnetic field to stimulate acupuncture points for treating various disorders. Additionally, the present invention provides magnetoelectric nanoparticles that may be useful to non-invasively treat certain neurological disorders. Furthermore, the invention provides magnetoelectric nanoparticles that are useful to identify sensory receptors responsible for acupuncture effects and a control placebo effect.

The magnetoelectric nanoparticles are nanoconverters of magnetic fields to intrinsic electric signals due to their strong magnetoelectric coupling. The magnetoelectric nanoparticles include a polar ferromagnetic metal nanoparticle core surrounded by a magnetoelectric coupling inducing shell. As a particular embodiment, the present invention provides a magnetoelectric nanoparticle which is CoFe₂O₄—BaTiO₃.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1C show MENs-MS induced neuronal activity in organotypic slice culture. After a coronal slice from Thyl-GCaMP6 transgenic mice was cultured for 10 days, MENs in artificial cerebrospinal fluid was added and a magnet was applied for 30 minutes in an incubator to load the MENs. Following two-photon imaging at baseline (FIG. 1A), application of MS (450 Oe at 10 Hz for 20 s) caused great increase in calcium fluorescence intensity in neuronal somata and dendrites (FIG. 1B), which decreased after turning of MS (FIG. 1C). n=4 slices.

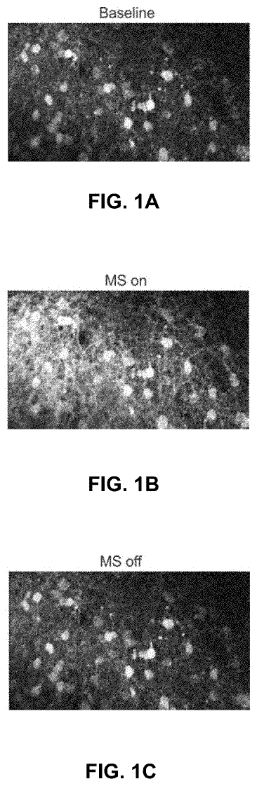

FIGS. 2A-2B show magnetic stimulation on MENs (MENs-MS) induced electrical potentials. FIG. 2A shows 595 nm channels and FIG. 2B shows 700 nm channels. An electrically sensitive fluorescent dye ANEPP was loaded with Ormosil particles for imaging electrical potential. The left panel in each of FIG. 2A and FIG. 2B shows low fluorescence of the 595 nm and 700 nm channels with magnetic wave (MS) only. The middle panel in each of FIG. 2A and FIG. 2B shows no fluorescence in both channels with MENs only. With MENs+MS, the right panel in each of FIG. 2A and FIG. 2B shows high fluorescence signal in both channels. The result suggest that a magnetic wave was able to induce electrical potentials. Scale bar: 5 μm.

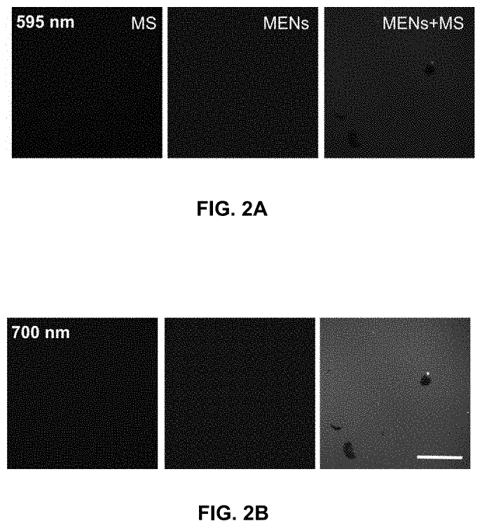

FIGS. 3A-3C show delivery of fMENs into mouse neocortex in vivo. Shown in FIG. 3A, in a live mouse, in vivo two-photon (2P) images of the cortex through a cranial window were taken before and after i.v. injection of saline (1^{st} column), 10 μg fMENs through retroorbital route (2^{nd} column), after applying 3000 Oe magnetic field on the mouse head for 45 minutes (3^{rd} column), and on the second day after fMENs injection (4^{th} column). The red fluorescence after magnetic field (MF) application (MENs+MF), but not MENs only, indicates successful delivery of MENs to cortical tissue only after applying magnetic force. Most of the delivered fMENs retained in the cortex after 24 hours. The Top row shows red fluorescent channel (fMEN) only, the bottom channel shows overlays of both green and red channels. Scale bar: 100 μm. Shown in FIG. 3B is a z-axis projection of a 2P image stack that shows the distribution of fMENs at different depths of the cortex. Shown in FIG. 3C is quantification of fluorescence intensity that shows increased red fluorescence after intravenous MEN injection followed by magnet application and retaining of a large percent of fluorescence signals at 24 hours after. n=3 mice.

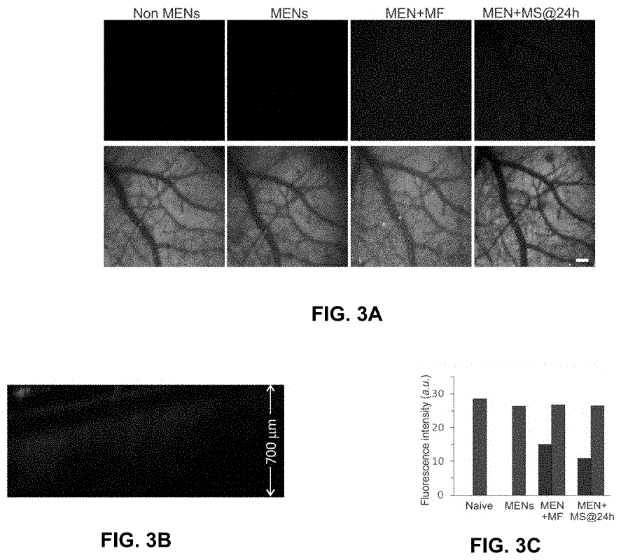

FIGS. 4A-4D show MENs-MS induced cortical neuronal activity in vivo. A mouse expressing calcium sensitive protein GCaMP6 was injected with 100 μl MEN solution through retroorbital vein, which was followed by applying a permanent magnet on one cortical surface to deliver MENs to a focal cortical area. As shown in FIGS. 4A and 4B, representative two-photon images in FIG. 4A were taken from a GCaMP6f mouse and changes in calcium signals in cortical layer II/III at baseline, after turning on MS (350 Oe at 10 Hz) for 30 s, and immediately after turning off MS were shown in FIG. 4B. The dotted color circles in FIG. 4A mark neuronal somata that show calcium traces in FIG. 4B with corresponding colors. Note that magnetic stimulation (MS) resulted neuronal activation as indicated by the increased fluorescence as calcium spikes. As shown in FIGS. 4C and 4D, significant increases in mean calcium spike amplitude (FIG. 4C) and number of spikes per minute (FIG. 4D) after turning on MS. (*: p<0.05, one-way ANOVA followed by post hoc test). Scale bar in FIG. 4A: 25 μm. n=5 mice for FIGS. 4C and 4D.

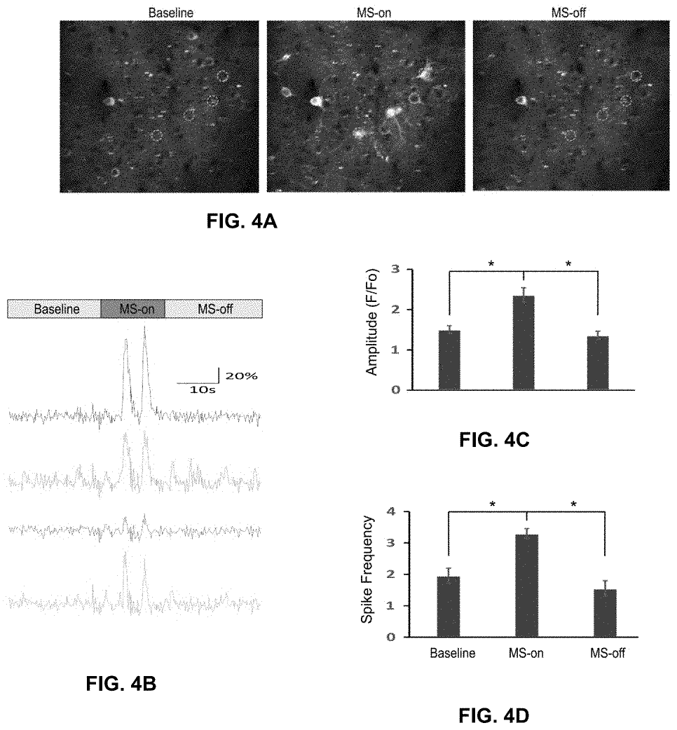

FIGS. 5A-5C show mesoscopic imaging of cerebral cortex showing activation of neurons in cortical hemispheres. Imaging was made through a large cranial window that expose both cortical hemispheres in a transgenic mouse expressing calcium sensitive protein GCaMP 6. Before imaging, the mouse was injected with 100 μl MEN solution through a retroorbital vein, which was followed by applying a pyramid permanent magnet on one cortical surface to attract MENs to the local area. As shown in FIG. 5A, fluorescence intensity increased after magnetic stimulation was turned on (middle image, MS-on, 450 Oe at 10 Hz for 30 s) and recovered immediately after the activation. As shown in FIG. 5B, mean fluorescence intensity was measured from cortical hemispheres that were ipsilateral (top) and contralateral (bottom) to the side of MENs delivery. As shown in FIG. 5C, there were significant increases in fluorescence intensity after magnetic stimulation on both cortical hemispheres. *: p<0.05, One-way ANOVA.

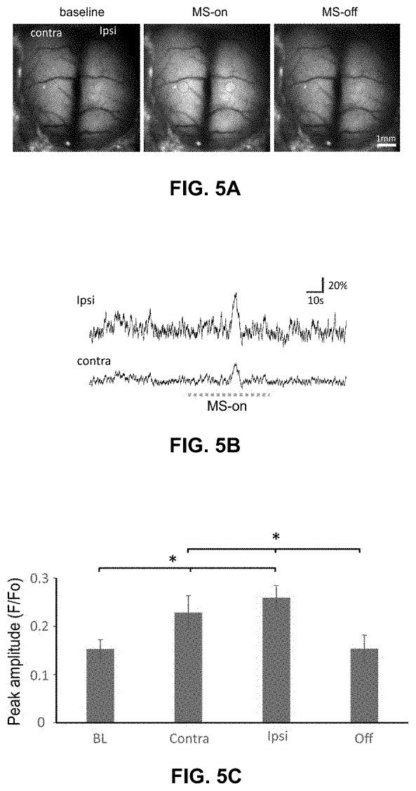

FIG. 6 shows magnetic stimulation of mice with local injection of MENs on an acupuncture point reduced pain sensitivity in a neuropathic pain model. A total of 20 C57BL mice received tibial nerve injury (TNI) as a model of neuropathic pain and waited for 7 days for the pain to develop. The mice were randomly assigned to two saline groups and two MENs groups that received a single inject of 50 μl of saline or MENs (20 mg/ml with GMO coating on nanoparticle surface) into an acupuncture point on leg (Yanglingquan, GB34). One from each of the saline or MEN groups further received magnetic stimulation (ms, 850 oe at 10 Hz, 6 sessions of 5 min stimulation with 2 minutes intervals in between). The result showed that the TNI surgery caused significant decrease in pain threshold as measured by von Frey tests at baseline and 7 days after TNI. Application of MS did not decrease pain sensitivity in the saline group, but caused a significant decrease in the MENs injection group, suggesting that magnetic stimulation with MENs in acupuncture point has analgesic effect. However, further MS on day 1 and day 3 did not have any effect on pain, likely because the MENs underneath the acupuncture point had been removed from the tissue. N=5 mice in each group. * p<0.05, One-way ANOVA followed by post hoc tests.

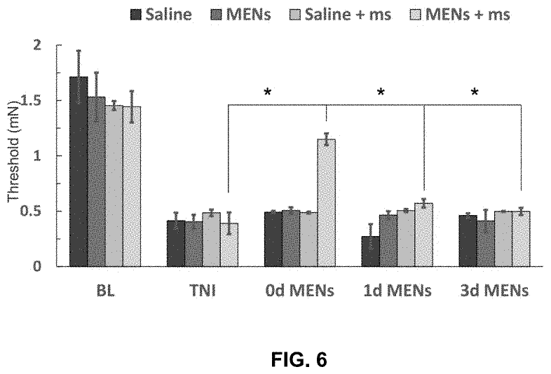

In embodiments of the invention, the magnetoelectric nanoparticles are configured to provide spatial and temporal control of their delivery for localized therapy. In embodiments of the present invention, the magnetoelectric nanoparticles are decorated with ligands or molecules that target to a specific type of cells or subcellular component in a target region of the subject. Preferred embodiments of targeting ligands or molecules include peptides, antibodies, aptamers, and proteins.

In embodiments of the present invention the magnetoelectric nanoparticles have a size of from about 5 nm to about 60 nm.

The present invention provides a pharmaceutical composition comprising a magnetoelectric nanoparticle, and a pharmaceutically acceptable carrier, diluent, or excipient. The present invention provides a pharmaceutical composition comprising CoFe₂O₄—BaTiO₃ and a pharmaceutically acceptable carrier, diluent, or excipient.

The present invention provides a method to treat a disorder, the method comprising: administering to a target region of a subject in need thereof an effective amount of a magnetoelectric nanoparticle, and, applying a magnetic field to the target region of the subject. In embodiments of the present invention, the administering step may be non-invasive or minimally invasive. The present invention provides a pharmaceutical composition for treating various disorders, the pharmaceutical composition comprising a magnetoelectric nanoparticle. The present invention provides a pharmaceutical composition for treating neurological disorders comprising a magnetoelectric nanoparticle and a pharmaceutically acceptable carrier, diluent, or excipient.

The present invention provides for administering a magnetoelectric nanoparticle to a target region of a subject. In embodiments of the present invention, the target region comprises different cell types in different locations of the body. In embodiments of the present invention, the target region is a cell type or receptor in a body location selected from one or more of the head, forehead, face, scalp, neck, upper chest, abdomen, upper back, lower back, hip, upper leg, knee, lower leg, ankle, foot, toe, shoulder, upper arm, elbow, forearm, wrist, hand, and finger. As a particular embodiment, the target region is selected from one or more acupuncture points on the human body that are known to one of ordinary skill in the art. Examples of acupuncture points include, but are not limited to, lung meridian points LU1-LU11, large intestine meridian points LI1-L120, stomach meridian points ST1-ST45, spleen meridian points SP1-SP21, heart meridian points HT1-HT9, small intestine meridian points SI1-S119, urinary bladder meridian points UB1-UB67, kidney meridian points KI1-127, pericardium meridian points P1-P9, triple warmer meridian points TW1-TW-23, and gall bladder meridian points GB1-44, points of eight extraordinary meridians, extra-meridiem points, trigger points, Ashi points, and points of the other acupuncture systems such as scalp acupuncture, auricular acupuncture. In embodiments of the invention, the MENs may be injected directly into individual acupuncture points. Alternatively, the MENs may be administered non-invasively, or even injected, and then moved to individual acupuncture points through the application of a magnetic field or an electric field.

Disorders that may be treated include any disorder that may be treated by conventional acupuncture. In embodiments of the present invention, the disorder is selected from one or more of migraine, headache, lower back pain, allergic rhinitis, knee osteoarthritis, nausea, vomiting, stroke asthma, pain, fatigue, constipation, depression, dry eye, hypertension, insomnia, irritable bowel syndrome, menopausal hot flashes, infertility, obesity, post-traumatic stress disorder, restless leg syndrome, schizophrenia, substance abuse, and sciatica. In addition, disorders that may be treated include any disorder associated with an organ system. Organ systems include, but are not limited to, the nervous system, the digestive system, the respiratory system, the circulatory system, the skeletal system, the integumentary system, the muscular system, the excretory system, the endocrine system, the reproductive system and the lymphatic/immune system. However, it should be understood that the disclosed methods may be applied to many other medical disorders, and that their application is not limited to the examples that are given. Furthermore, this invention may also be applied for other indications of acupuncture treatment, such as acupuncture facial rejuvenation and smoking cessation.

The present invention provides for externally applying a magnetic field to a target region of the subject. In embodiments of the invention, the magnetic field generator is one or more electromagnets. The target anatomical region may be positioned to receive the magnetic field. In embodiments of the invention, a magnetic field generator is positioned non-invasively on or above or near a target anatomical region, such as on or near the neck, ankle, abdomen or scalp, or in the vicinity of nerves or tissue that control a physiological reflex or response. Alternatively, the magnetic field generator may be positioned while the target anatomical region is stationary. In other embodiments, both the magnetic field generator and the target region may be positioned to achieve a desired result. In embodiments of the present invention, the magnetic field is applied more than once to the target region, i.e., the magnetic field is pulsed. In other embodiments, the magnetic field is applied continuously to the target region. In embodiments of the present invention, the magnetic field is generated by an electromagnetic coil. In embodiments of the present invention, the magnetic field is a directed magnetic field. In embodiments of the present invention, the magnetic field is an AC magnetic field. In embodiments of the present invention, the magnetic field is a low energy magnetic field.

The present invention provides for externally applying a magnetic field to a target region of the subject. In embodiments of the present invention, the strength of the magnetic field is in the range of from about 100 Oe to about 3000 Oe.

In embodiments of the present invention, the frequency of the magnetic field is in the range of from about 5 Hz to about 1000 Hz.

In embodiments of the present invention, the duration of the magnetic field is in the range of from about 1 s to about 60 s.

In embodiments of the present invention, the administered magnetoelectric field and the externally applied magnetic field interact to generate an electric field in proximity to the target region of the subject. In one embodiment, the generated electric field is greater than about 1000 V/m. In a preferred embodiment, the electric field can be generated remotely/wirelessly.

As used above, and throughout the description of the invention, the following terms, unless otherwise indicated, shall be understood to have the following meanings:

A “pharmaceutically acceptable carrier, diluent, or excipient” is a medium generally accepted in the art for the delivery of biologically active agents to mammals, e.g., humans.

“Effective amount” means the amount of the magnetoelectric nanoparticle, of the present invention or pharmaceutical composition containing a magnetoelectric nanoparticle, of the present invention that will elicit the biological or medical response of or desired therapeutic effect on a tissue, system, animal, mammal or human that is being sought by the researcher, veterinarian, medical doctor or other clinician.

The terms “treatment,” “treat,” “treating,” and the like, are meant to include slowing or reversing the progression of a disorder. These terms also include alleviating, ameliorating, attenuating, eliminating, or reducing one or more symptoms of a disorder or condition, even if the disorder or condition is not actually eliminated and even if progression of the disorder or condition is not itself slowed or reversed.

The magnetoelectric nanoparticle of the present invention is preferably formulated as a pharmaceutical composition using a pharmaceutically acceptable carrier, diluent, or excipient and administered by a variety of routes. Such compositions can be formulated and delivered via various routes of administration such as intravenous, intramuscular, subcutaneous injection, oral administration and inhalation. Compositions for parenteral administration can be in the form of aqueous or non-aqueous isotonic sterile injection solutions or suspensions. Such pharmaceutical compositions and processes for preparing them are well known in the art. See, e.g., Remington: The Science and Practice of Pharmacy (A. Gennaro, et al., eds., 21st ed., Mack Publishing Co., 2005).

The amount of the magnetoelectric nanoparticle of the present invention actually administered will be determined by a practitioner under the relevant circumstances, including the condition to be treated, the chosen route of administration, the actual magnetoelectric nanoparticle of the present invention administered, the age, weight, and response of the individual patient, and the severity of the patient's symptoms. Dosages per day normally fall within the range of about 1 to about 1000 mg. In some instances, dosage levels below the lower limit of the aforesaid range may be more than adequate, while in other cases still larger doses may be employed. Dosage levels can be determined by one of skill in the art.

The magnetoelectric nanoparticles of the present invention may be prepared by a variety of procedures known in the art, as well as those described in the Examples below. The specific synthetic steps for each of the routes described may be combined in different ways to prepare the magnetoelectric nanoparticles of the invention.

The magnetoelectric nanoparticles of the present invention can be prepared according to synthetic methods well known and appreciated in the art. The reagents and starting materials are generally readily available to one of ordinary skill in the art. Others may be made by standard techniques of chemistry, techniques that are known to one of ordinary skill in the art, and the procedures described in the Examples that follow including any novel procedures.

### EXAMPLE 1

A compound within the scope of the invention was tested substantially as described above. The compound activated neurons as follows. A coronal brain slice from a GCaMP6 transgenic mouse was cultured for 10 days. Neuronal expression of calcium sensitive protein GCaMP6s allowed us to directly see neurons that became active. Artificial cerebrospinal fluid containing MENs was added to the slice and a magnet was applied for 30 minutes in an incubator. After magnetic wave (10 Hz, 450 Oe) was turned on, fluorescence in many neurons in the slice was increased after a delay and the background fluorescence also increased, indicating stimulated neuronal activity.

Various modifications and additions can be made to the embodiments disclosed herein without departing from the scope of the disclosure. For example, while the embodiments described above refer to particular features, the scope of this disclosure also includes embodiments having different combinations of features and embodiments that do not include all of the described features. Thus, the scope of the present disclosure is intended to embrace all such alternatives, modifications, and variations as fall within the scope of the claims, together with all equivalents.

All publications, patents and patent applications referenced herein are hereby incorporated by reference in their entirety for all purposes as if each such publication, patent or patent application had been individually indicated to be incorporated by reference.

1. A method to treat a disorder, the method comprising: administering to a target region of a subject in need thereof an effective amount of a type of magnetoelectric nanoparticles, and externally applying a magnetic field to the target region of the subject.

2. The method of claim 1, wherein the administering step is non-invasive or minimally invasive.

3. The method of claim 1, comprising CoFe₂O₄—BaTiO₃ or other type of magnetoelectric nanoparticles

4. The method of claim 1, further comprising a pharmaceutically acceptable carrier.

5. The method of claim 1, wherein the externally applied magnetic field is an externally applied directed magnetic field.

6. The method of claim 1, wherein the administered magnetoelectric nanoparticles and the externally applied magnetic field interact to generate an electric field in proximity to tissues and cells in the target region of the subject.

7. The method of claim 6, wherein the generated electric field is greater than about 1000 V/m.

8. A method to stimulate a cell type or receptor-containing region in a subject or an experimental animal that is associated with an acupuncture response, the method comprising: administering to a target region of a subject in need thereof an effective amount of a magnetoelectric nanoparticle, and externally applying a magnetic field to the target region of the subject.

9. The method of claim 8, wherein the method is non-invasive or minimally invasive.
